(19) United States
(12) Patent Application Publication
(10) Pub. No.: US 2015/0320489 A1
(43) Pub. Date: Nov. 12, 2015

(54) ABLATION PROBE FIXATION

(71) Applicant: COVIDIEN LP, MANSFIELD, MA (US)

(72) Inventors: DARION PETERSON, BOULDER, CO (US); JOSEPH D. BRANNAN, ERIE, CO (US); KENLYN S. BONN, LAKEWOOD, CO (US); RICHARD W. WETZ, ERIE, CO (US); FRANCESCA ROSSETTO, LONGMONT, CO (US); IAN SMITH, BOULDER, CO (US)

(21) Appl. No.: 14/806,014

(22) Filed: Jul. 22, 2015

Related U.S. Application Data
(63) Continuation of application No. 12/493,302, filed on Jun. 29, 2009, now abandoned.

(51) Int. Cl. A61B 18/14; A61B 19/00

(52) U.S. Cl. CPC A61B 18/1477; A61B 19/26; A61B 2018/1869

(57) ABSTRACT

An ablation probe fixation apparatus for securing an ablation probe to tissue includes a base having a top surface and a skin-contacting bottom surface, wherein the base includes an adhesive layer disposed on the skin-contacting bottom surface. The fixation apparatus also includes a fixation member coupled to the top surface of the base. The base and the fixation member include an aperture defined therein for insertion of the ablation probe therethrough.

### CROSS-REFERENCE TO RELATED APPLICATION

The present application is a continuation of U.S. patent application Ser. No. 12/493,302, filed on Jun. 29, 2009, the contents of which is incorporated by reference herein in its entirety.

### BACKGROUND

1. Technical Field

The present disclosure relates generally to ablation probes used in tissue ablation procedures. More particularly, the present disclosure is directed to a system and method for fixating the ablation probe to tissue.

2. Background of Related Art

Therapeutic lesions in living bodies have been accomplished for many decades using radio-frequency (RF) and other forms of energy. The procedures have been particularly useful in the field of neurosurgery and tumor necrosis. Such methods involve applying electromagnetic radiation to heat tissue and include ablation and coagulation of tissue. Various types of ablation probes may be utilized to heat tissue to the desired temperature, such as microwave, electrosurgical, and resistive heating. Typically, ablation electrodes (usually of elongated cylindrical geometry) are inserted into a living body (percutaneously or during an open procedure) and energy is applied thereto. A typical form of such ablation electrodes incorporates an insulated sheath from which an exposed (uninsulated) tip extends.

### SUMMARY

According to one aspect of the present disclosure, an ablation probe fixation apparatus for securing an ablation probe to tissue is disclosed. The ablation probe fixation apparatus includes a base having a top surface and a skin-contacting bottom surface, wherein the base includes an adhesive layer disposed on the skin-contacting bottom surface. The fixation apparatus also includes a fixation member coupled to the top surface of the base. The base and the fixation member include an aperture defined therein for insertion of the ablation probe therethrough.

According to another aspect of the present disclosure, an ablation probe fixation apparatus for securing an ablation probe to tissue is disclosed. The ablation probe fixation apparatus includes an adhesive amorphous putty adapted to be perforated by an ablation probe. The adhesive amorphous putty configured to be shaped from a first configuration into a subsequent configuration for securing the ablation probe therein.

A method for securing an ablation probe to tissue is also contemplated by the present disclosure. The method includes the steps of: applying an ablation probe fixation apparatus to the tissue, the fixation apparatus being formed from an adhesive amorphous putty adapted to be perforated by the ablation probe. The method also includes the steps of shaping the adhesive amorphous putty from a first configuration into a subsequent configuration for securing the ablation probe therein and inserting the ablation probe through the fixation apparatus into the tissue.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1 is a schematic diagram of an ablation system according to an embodiment of the present disclosure;

FIG. 2 is a perspective view of an ablation probe according to an embodiment of the present disclosure;

FIG. 3A is a perspective view of an ablation probe fixation apparatus according to an embodiment of the present disclosure showing a single-camming mechanism;

FIG. 3B is a side, cross-sectional view of the fixation apparatus of FIG. 3A according to an embodiment of the present disclosure;

FIG. 3C is a side, cross-sectional view of an ablation probe and the fixation apparatus of FIG. 3A;

FIG. 4 is a side, cross-sectional view of an ablation probe fixation apparatus according to an embodiment of the present disclosure showing a double-camming mechanism;

FIG. 5A is a side, cross-sectional view of an ablation probe fixation apparatus according to an embodiment of the present disclosure showing a clamping mechanism;

FIG. 5B is a top view of the fixation apparatus of FIG. 5A;

FIG. 6A is a perspective view of an ablation probe fixation apparatus according to an embodiment of the present disclosure showing a fixation member;

FIG. 6B is a side, cross-sectional view of the fixation apparatus of FIG. 6A;

FIG. 7A is a perspective view of an ablation probe fixation apparatus according to an embodiment of the present disclosure showing fastening elements;

FIG. 7B is a side, cross-sectional view of the fixation apparatus of FIG. 7A;

FIG. 8A is a perspective view of an ablation probe fixation apparatus according to an embodiment of the present disclosure showing a half-shell member;

FIG. 8B is a side, cross-sectional view of the fixation apparatus of FIG. 8A;

FIG. 9A is a perspective view of an ablation probe fixation apparatus according to an embodiment of the present disclosure showing a gel diaphragm;

FIG. 9B is a side, cross-sectional view of the fixation apparatus of FIG. 9A;

FIG. 10A is a perspective view of an ablation probe fixation apparatus according to an embodiment of the present disclosure showing a putty;

FIG. 10B is a side, cross-sectional view of the fixation apparatus of FIG. 10A;

FIG. 11 is a perspective view of an ablation probe according to an embodiment of the present disclosure showing a deployable member;

FIG. 12 is a perspective view of an ablation probe according to an embodiment of the present disclosure showing a deployable member;

FIG. 13A is a side view of an ablation probe fixation apparatus according to an embodiment of the present disclosure showing a clamp member;

FIG. 13B is top view of the fixation apparatus of FIG. 13A;

FIG. 14 is a side view of an ablation probe fixation apparatus according to an embodiment of the present disclosure showing an arm member;

FIG. 15A is a perspective view of an ablation probe fixation apparatus according to an embodiment of the present disclosure showing a magnetic member;

FIG. 15B is a side, cross-sectional view of the fixation apparatus of FIG. 15A;

FIG. 16A is a perspective view of an ablation probe fixation apparatus according to an embodiment of the present disclosure showing an electromagnetic member; and

FIG. 16B is a side, cross-sectional view of the fixation apparatus of FIG. 16A.

### DETAILED DESCRIPTION

Particular embodiments of the present disclosure are be described herein below with reference to the accompanying drawings. In the following description, well-known functions or constructions are not described in detail to avoid obscuring the present disclosure in unnecessary detail.

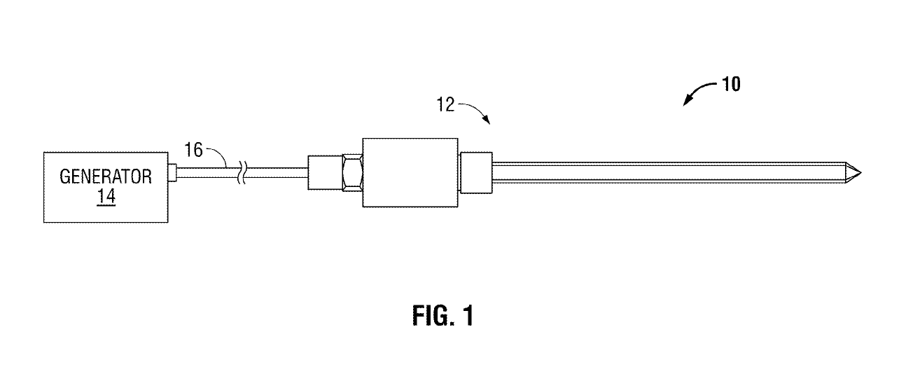

FIG. 1 shows an ablation system 10 that includes an ablation probe 12 coupled to a generator 14 via a cable 16. The generator 14 is configured to provide electromagnetic energy (e.g., high frequency electrosurgical energy and/or microwave energy at an operational frequency from about 100 kHz to about 10,000 MHz). The ablation probe 12 may be any type of probe suitable for delivering energy to tissue, such as an electrosurgical or microwave probe. During use, one or more probes 12 are inserted into target tissue (e.g., tumor) to a predetermined depth, such that when energy is applied thereto an ablation volume is created suitable to destroy the target tissue. It is desirable to maintain the placement of the probe 12 in the target tissue and prevent displacement due to various disturbances (e.g., movement of the patient, patient respiration, etc.).

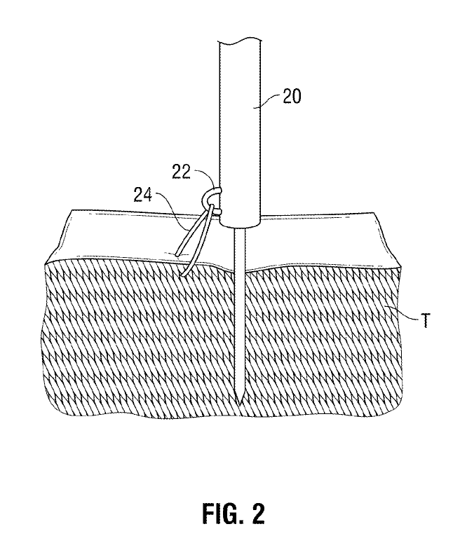

FIGS. 2-16B illustrate various embodiments of fixating the probe 12 to the patient to reduce or eliminate probe displacement. With reference to FIG. 2, an ablation probe 20 is shown having one or more tie-down features 22. The ablation probe 20 is adapted to be inserted into the tissue “T” and thereafter secured to the tissue “T” via a suture 24. In one embodiment, the suture 24 may be any type of thread, string, wire and the like. The feature 22 may be a loop, a hook or any other type of protrusion suitable for being tied to the suture. More specifically, the suture 24 may be passed through or otherwise secured to the feature 22 and thereafter the suture is stitched to the tissue “T,” thereby securing the probe 20 thereto.

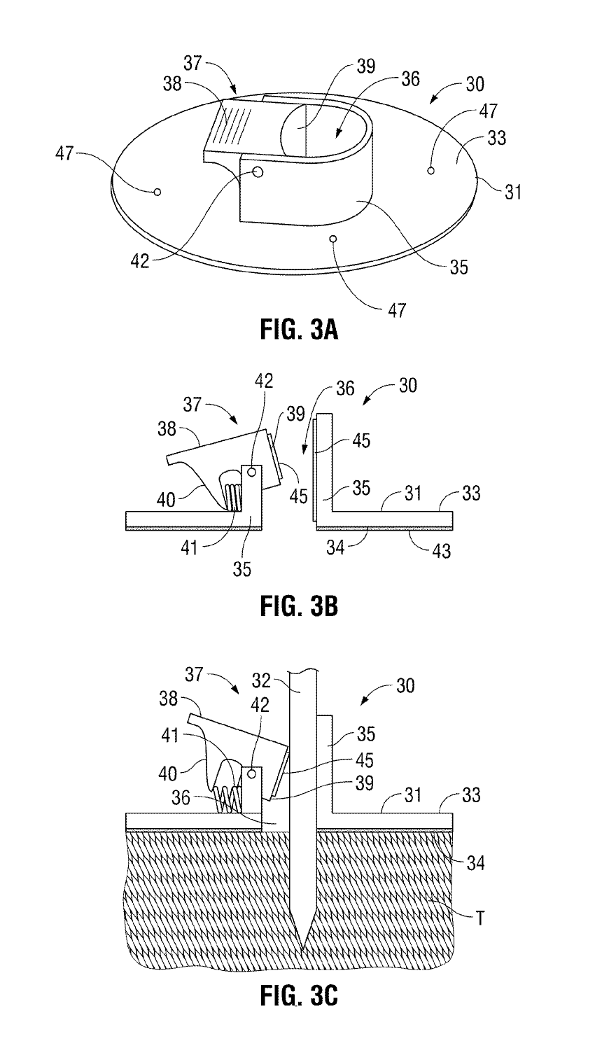

FIGS. 3A-3C show an ablation probe fixation apparatus 30 for securing an ablation probe 32 (FIG. 3C) within the tissue “T.” With reference to FIGS. 3A-3B, the apparatus 30 includes a base 31 having a top surface 33 and a skin-contacting bottom surface 34. The base 31 may have any suitable shape such as oval, round, rectangular, polygonal, etc. The base 31 includes an adhesive layer 43 disposed on the bottom surface 34 thereof as shown in FIG. 3B. The apparatus 30 also includes a support shaft 35 defining an aperture 36 therethrough (FIG. 3A). The shaft 35 may partially encircle the probe 32 allowing the probe 32 to be inserted through the aperture 36. The shaft 35 may be integral with the base 31 or may be formed from a separate structure and then attached thereto. As shown in FIGS. 3B and 3C, the shaft 35 is disposed transversely with respect to the base 31. In one embodiment, the shaft 35 may be disposed at any angle with respect to the base 31 allowing for the insertion path of the probe 32 into the tissue “T” to substantially match the angle between the base 31 and the shaft 35.

With reference to FIG. 3A, the apparatus 30 also includes a camming member 37 pivotally coupled to the shaft 35 via a pivot pin 42. The camming member 37 includes a lever 38 at one end and a camming surface 39 at another end. In one embodiment, the camming surface 39 and the inside surface of the shaft 35 may include a high friction surface 45 (not explicitly shown on the shaft 35). The surface 45 may be formed from a high friction compressible material (e.g., rubber, foam, etc.) to lessen the force applied to the probe 32 and may also include an adhesive layer to provide additional fixation reliability. As shown in FIGS. 3B and 3C, the camming member 37 further includes a protrusion 40 extending downward therefrom. The protrusion 40 is biased by a biasing member 41 (e.g., spring) disposed between the protrusion 40 and the shaft 35.

During operation, the apparatus 30 is secured against the tissue “T” via the adhesive layer 43. In one embodiment, a protective film may be disposed over the adhesive layer 43 to protect the adhesive prior to use. Thereafter, the camming member 37 is pushed downward from a closed configuration (FIG. 3C) to an open configuration (FIG. 3B) in a counterclockwise direction about the pivot pin 42, thereby opening the aperture 36 and allowing the probe 32 to be inserted therethrough into the tissue “T.” Once the probe 32 is in a desired location, the force pushing down on the camming member 37 is removed, and the camming member 37 returns in a clockwise direction about the pivot pin 42 to the closed configuration and engages the probe 32 (FIG. 3C). This secures the probe 32 between the camming member 37 and the inside surface of the shaft 35.

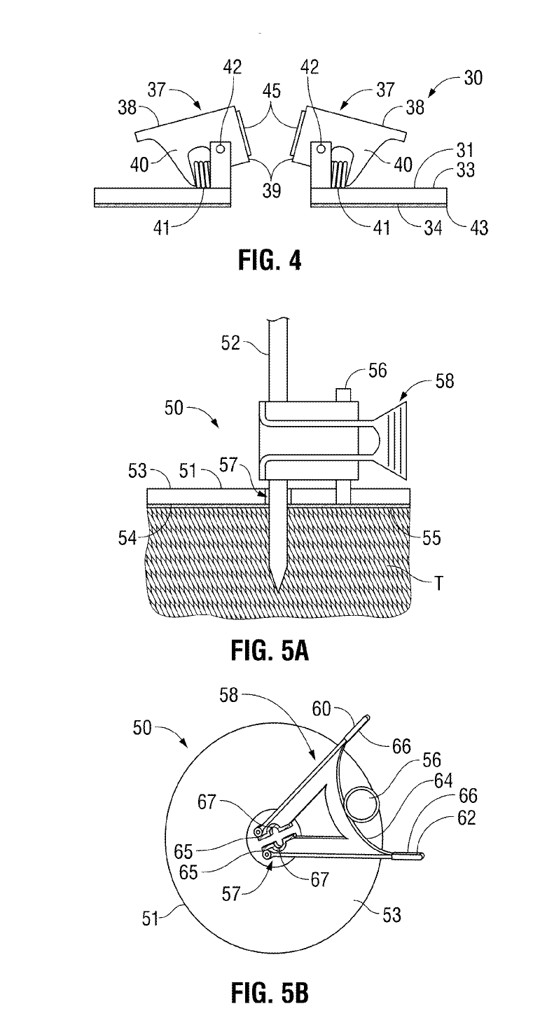

In one embodiment, the apparatus 30 may include multiple shafts 35 and corresponding camming members 37 to provide for insertion and fixation of multiple probes 32. In another embodiment as shown in FIG. 4, the shaft 35 may be replaced by another camming member 37 to provide two opposing camming members 37 on either side of the aperture 36. The opposing camming members 37 may be linked (e.g., lever, wire, etc.) to a single button or lever (not explicitly shown) to provide for simultaneous opening and closing of the opposing camming members 37.

In a further embodiment, the apparatus 30 may include one or more skin temperature monitoring devices 47, such as thermal probes, thermocouples, thermistors, optical fibers and the like, to monitor skin surface temperature as shown in FIG. 3A.

FIGS. 5A-5B show an ablation probe fixation apparatus 50 for securing an ablation probe 52 within the tissue “T” (FIG. 5A). The apparatus 50 includes a base 51 having a top surface 53 and a skin-contacting bottom surface 54. The base 51 may have any suitable shape such as oval, round, rectangular, polygonal, etc. The base 51 also includes an adhesive layer 55 disposed on the bottom surface 54 thereof as shown in FIG. 5A. The apparatus 50 also includes a fixation post 56 and aperture 57 defined therein (FIG. 5B). The post 56 may be integral with the base 51 or may be formed from a separate structure and then attached thereto.

With reference to FIG. 5B, the apparatus 50 also includes a clamp 58 coupled to the post 56. The clamp 58 includes two opposing levers 60 and 62 having a biasing member 64 therebetween. Each of the opposing levers 60 and 62 include a distal end 65 and a proximal end 66. The biasing member 64 forces the levers 60 and 62 closed at the distal ends 65 and open at the proximal ends 66. Each of the levers 60 and 62 include a high friction surface 67 disposed at the distal ends 65. The high friction surface 65 may be formed from a high friction compressible material (e.g., rubber, foam, etc.) to lessen the force applied to the probe 52 and may also include an adhesive layer to provide additional fixation reliability. The clamp 58 is coupled to the post 56 such that the distal ends 65 are disposed over the aperture 57.

During operation, the apparatus 50 is secured against the tissue “T” via the adhesive layer 55. In one embodiment, a protective film may be disposed over the adhesive layer 55 to protect the adhesive prior to use. Thereafter, the levers 60 and 62 are pushed together at the proximal ends 66 to open at the distal ends 65 allowing the probe 52 to be inserted in between the levers 60 and 62 and through the aperture 57 into the tissue “T” (FIG. 5A). Once the probe 52 is in a desired location, the force on the proximal ends 66 of the levers 60 and 62 is removed, and the levers 60 and 62 clamp the probe 52 therebetween (FIG. 5A).

In one embodiment, the apparatus 50 may include multiple posts 56 and corresponding clamps 58 to provide for insertion and fixation of multiple probes 52. In another embodiment, the apparatus 50 may include one or more skin temperature monitoring devices, such as thermal probes, thermocouples, thermistors, optical fibers and the like, to monitor skin surface temperature (FIG. 3A).

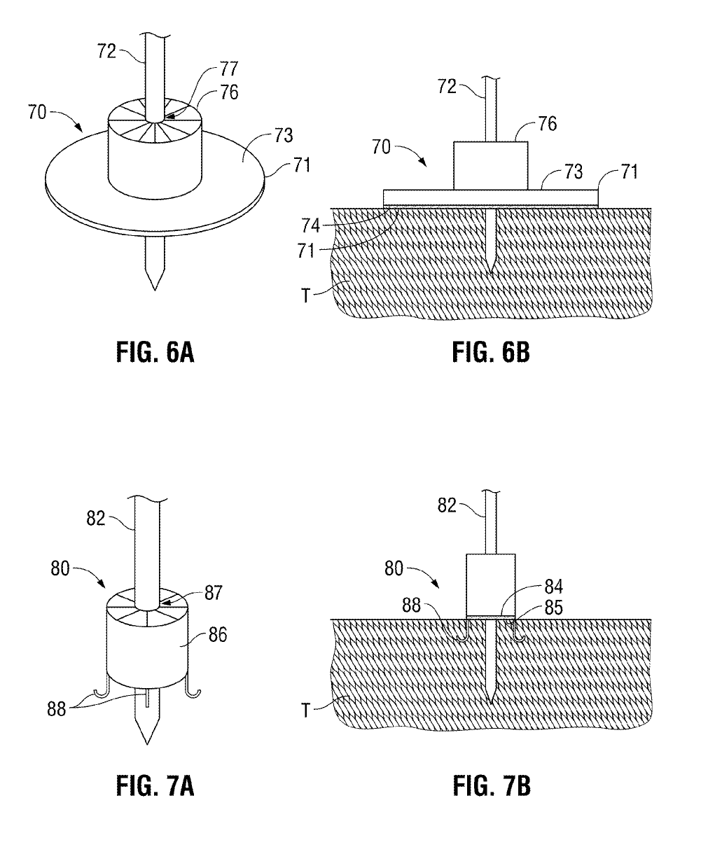

FIGS. 6A-6B show an ablation probe fixation apparatus 70 for securing an ablation probe 72 within the tissue “T” (FIG. 6A). The apparatus 70 includes a base 71 having a top surface 73 and a skin-contacting bottom surface 74. The base 71 may have any suitable shape such as oval, round, rectangular, polygonal, etc. The base 71 also includes an adhesive layer 75 disposed on the bottom surface 74 thereof as shown in FIG. 6B. In one embodiment, a protective film may be disposed over the adhesive layer 43 to protect the adhesive prior to use.

As shown in FIG. 6A, the apparatus 70 also includes a fixation member 76 defining an aperture 77 for insertion of the probe 72 therethrough and into the tissue “T.” The aperture 77 is sized to be in frictional contact with the probe 72 thereby preventing movement of the probe 72 while allowing for relatively easier insertion therethrough. The fixation member 76 is formed from any type of an elastomer to provide for frictional interface with the probe 72. The fixation member 76 may be integral with the base 71 or may be formed from a separate structure and then attached thereto. In one embodiment, the apparatus 70 may include one or more skin temperature monitoring devices, such as thermal probes, thermocouples, thermistors, optical fibers and the like, to monitor skin surface temperature.

FIGS. 7A-7B show an ablation probe fixation apparatus 80 for securing an ablation probe 82 within the tissue “T” (FIG. 7A). As shown in FIG. 7A, the apparatus 80 includes a fixation member 86 defining an aperture 87 for insertion of the probe 82 therethrough and into the tissue “T.” The aperture 87 is sized to be in frictional contact with the probe 82 thereby preventing movement of the probe 82 while allowing for relatively easier insertion therethrough. The fixation member 86 may be formed from any type of an elastomer to provide for frictional interface with the probe 82. The fixation member 86 also includes one or more fastening elements 88 disposed on a skin-contacting bottom surface 84. The elements 88 may be hooks, barbs and other tissue-penetrating elements suitable for retaining the fixation member 86. The fixation member 86 may also include an adhesive layer 85 disposed on the bottom surface 84 thereof as shown in FIG. 7B. In one embodiment, a protective film may be disposed over the adhesive layer 43 to protect the adhesive prior to use. In another embodiment, the apparatus 80 may include one or more skin temperature monitoring devices, such as thermal probes, thermocouples, thermistors, optical fibers and the like, to monitor skin surface temperature (FIG. 3A).

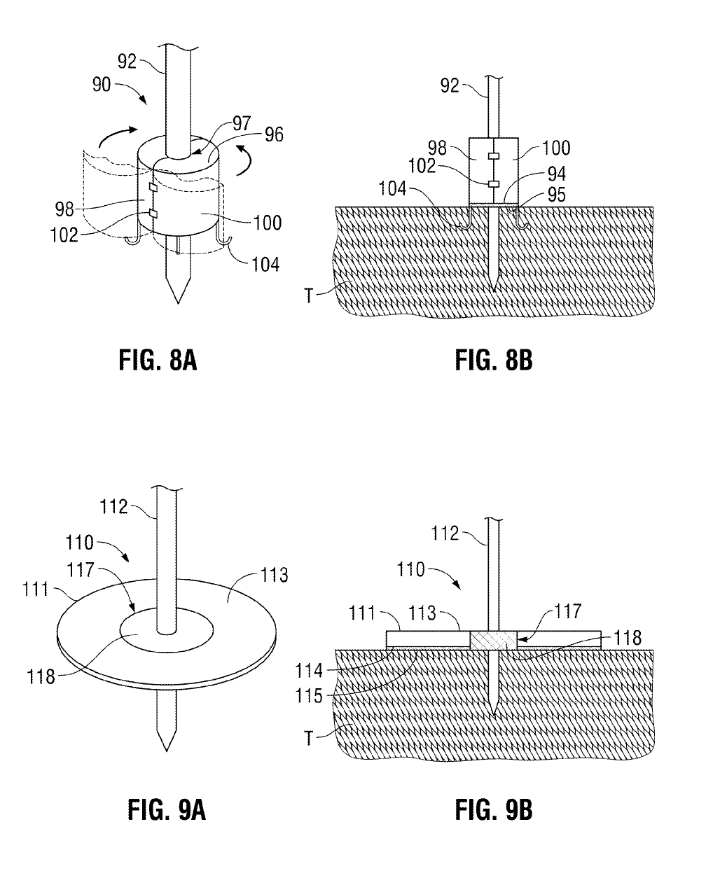

FIGS. 8A-8B show an ablation probe fixation apparatus 90 for securing an ablation probe 92 within the tissue “T” (FIG. 8A). As shown in FIG. 8A, the apparatus 90 includes a fixation member 96 defining an aperture 97 for insertion of the probe 92 therethrough and into the tissue “T.” The fixation member 96 includes a first half-shell 98 and a second half-shell 100 joined together by a hinge 102 (e.g., a living hinge). The first and second half-shells 98 and 100 are movable from a first position in spaced relation relative to one another for placing the probe 92 therebetween to a closed position for securing the probe 92 between the two half-shells 98 and 100.

The first and second half-shells 98 and 100 may include a high friction surface (not explicitly shown) around the aperture 97. The high friction surface may be formed from a compressible material (e.g., rubber, foam, etc.) to lessen the force applied to the probe 92. The aperture 97 may also include an adhesive layer to provide additional fixation reliability of the probe 92 to the fixation member 96.

The fixation member 96 also includes one or more fastening elements 104 disposed on a skin-contacting bottom surface 94. The elements 104 may be hooks, barbs and other tissue penetrating elements suitable for penetrating tissue and securing the fixation member 96 to the tissue “T.” The fixation member 96 may also include an adhesive layer 95 disposed on the bottom surface 94 thereof as shown in FIG. 8B. In one embodiment, a protective film may be disposed over the adhesive layer 43 to protect the adhesive prior to use.

During operation, the first and second half-shells 98 and 100 are opened and the probe 92 is placed therebetween. The half-shells 98 and 100 are then closed, and the fixation member 96 along with the probe 92 is inserted into the tissue “T” until the fastening elements 104 have penetrated the tissue “T.” In one embodiment, the apparatus 90 may include one or more skin temperature monitoring devices, such as thermal probes, thermocouples, thermistors, optical fibers and the like, to monitor skin surface temperature (FIG. 3A).

FIGS. 9A-9B show an ablation probe fixation apparatus 110 for securing an ablation probe 112 within the tissue “T” (FIG. 9A). The apparatus 110 includes a base 111 having a top surface 113 and a skin-contacting bottom surface 114 (FIG. 9B). The base 111 may have any suitable shape such as oval, round, rectangular, polygonal, etc. The base 111 also includes an adhesive layer 115 disposed on the bottom surface 114 thereof as shown in FIG. 9B.

As shown in FIG. 9A, the apparatus 110 also includes an aperture 117 defined therein for insertion of the probe 112 therethrough and into the tissue “T.” The aperture 117 includes a gel diaphragm 118 therein. The diaphragm 118 may be formed from various types of hydrogels or adhesives. In one embodiment, the diaphragm 118 may have an opening (not explicitly shown) defined therein. In another embodiment, the diaphragm 118 may be contiguous such that the probe 112 perforates the diaphragm 118 during insertion. The gel and/or adhesives of the diaphragm 118 maintain the probe 112 at the desired depth thereby preventing displacement of the probe 112 caused by patient movement. In one embodiment, the apparatus 110 may include one or more skin temperature monitoring devices, such as thermal probes, thermocouples, thermistors, optical fibers and the like, to monitor skin surface temperature (FIG. 3A).

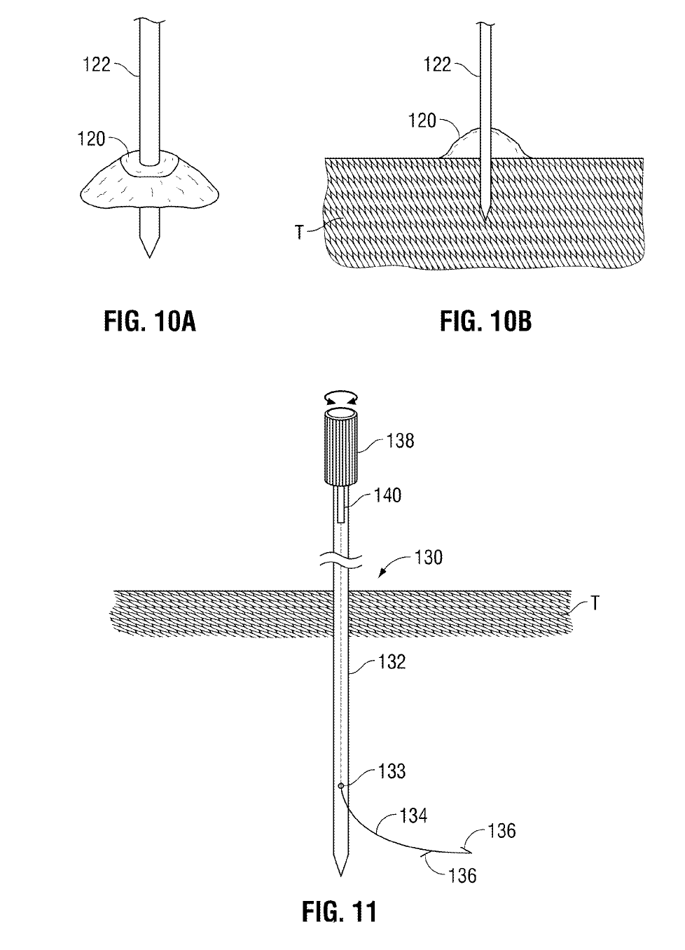

FIGS. 10A-10B show an ablation probe fixation apparatus 120 for securing an ablation probe 122 within the tissue “T” (FIG. 10A). The apparatus 120 is formed from an adhesive amorphous putty that may be molded under pressure but is still capable of retaining its shape. In other words, the putty may be shaped from a first configuration into a subsequent configuration for securing the ablation probe therein. In one embodiment, the amorphous putty may be a viscoelastic polymer composition having a siloxane polymer, a crystalline material and one or more thixotropic agents to reduce liquid properties thereof and enable the amorphous putty to hold its shape.

During use, the apparatus 120 is placed onto the tissue “T” and the probe 122 is inserted therethrough perforating the apparatus 120. The viscoelastic properties of the apparatus 120 allow the probe 122 to easily penetrate therethrough and into the tissue “T” as shown in FIG. 10B. Since the putty of the apparatus 120 is adhesive, the putty secures the apparatus 120 to the tissue “T” and maintains the position of the probe 122 therein.

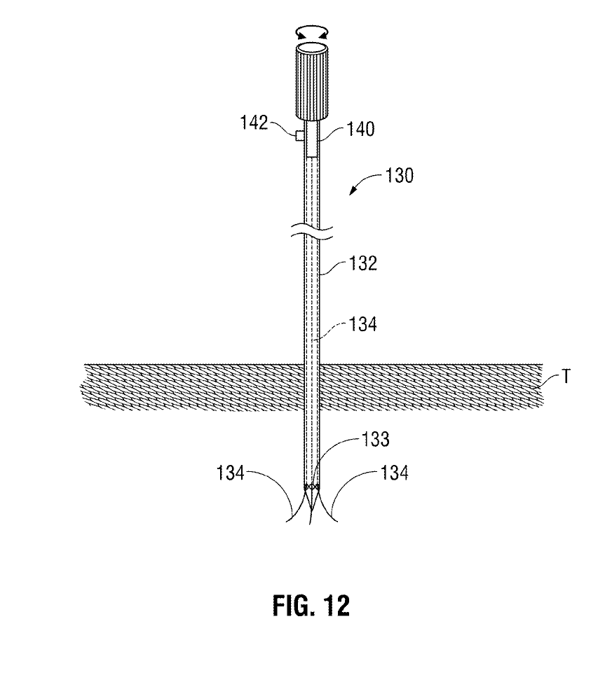

FIGS. 11 and 12 show an ablation probe 130 according to one embodiment of the present disclosure. The probe 130 includes a shaft 132 along which energy is communicated into the tissue “T.” The probe 130 includes one or more deployable fixation elements 134 disposed within the shaft 132 that are deployed through one or more corresponding openings 133. The fixation elements 134 are deployed once the shaft 132 is inserted into the tissue “T” to the desired depth to secure the probe 130 therein.

The fixation elements 134 may be expanding tines, hooks, barbs and the like. The fixation elements 134 may be formed from a flexible non-metallic material such that the fixation elements 134 do not interfere with the application of electromagnetic energy supplied through the shaft 132. The fixation elements 134 may be deployed along any portion of the shaft 132, such as shown in FIG. 11 or at a tip thereof as shown in FIG. 12.

With reference to FIG. 11, the fixation elements 134 also include one or more barbs 136 along the length thereof. The barbs 136 may be formed from the same material as the fixation elements 134. In one embodiment the barbs 136 may be formed from bimetallic strips that are flush with the fixation elements 134. The barbs 136 may then be activated by heating or supplying electrical current to expand from the fixation element 134 and form barb-like structures. In another embodiment, the barbs 136 may be formed from a bimetallic composition that becomes brittle during the ablation process allowing the barbs 136 to detach easily from the fixation element 134 upon retraction of the probe 130. In a further embodiment, the probe 130 may include one or more skin temperature monitoring devices within or on the fixation elements 134, such as thermal probes, thermocouples, thermistors, optical fibers and the like, to monitor skin surface temperature (FIG. 3A).

In one embodiment, the fixation elements 134 may be deployed by actuation of a rotational actuation knob 138 as shown in FIG. 11. The knob 138 is rotatable about a longitudinal axis as defined by the shaft 132. The knob 138 is coupled to a drive rod 140 disposed within the shaft 132. The drive rod 140 is coupled to one or more of the fixation elements 134. The knob 138 may be rotated in either clockwise or counter-clockwise direction, wherein rotation in one direction withdraws the fixation elements 134 and in the opposite direction deploys the fixation elements 134. More specifically, the rotational motion of the actuation knob 138 is translated into longitudinal motion of the drive rod 140, which then withdraws or deploys the fixation elements 134.

In another embodiment, the fixation elements 134 may be deployed by actuation of a slidable actuation knob 142 as shown in FIG. 12. The knob 142 is slidable along the longitudinal axis of the shaft 132 and is coupled to the drive rod 140 disposed within the shaft 132. The drive rod 140 is coupled to one or more of the fixation elements 134. The knob 142 may be slid in either a distal direction, toward the tip of the shaft 132, or in a proximal direction. Movement of the knob 142 in the proximal direction deploys the fixation elements 134 and movement in distal direction withdraws the fixation elements 134. The probe 132 is secured within the tissue “T.”

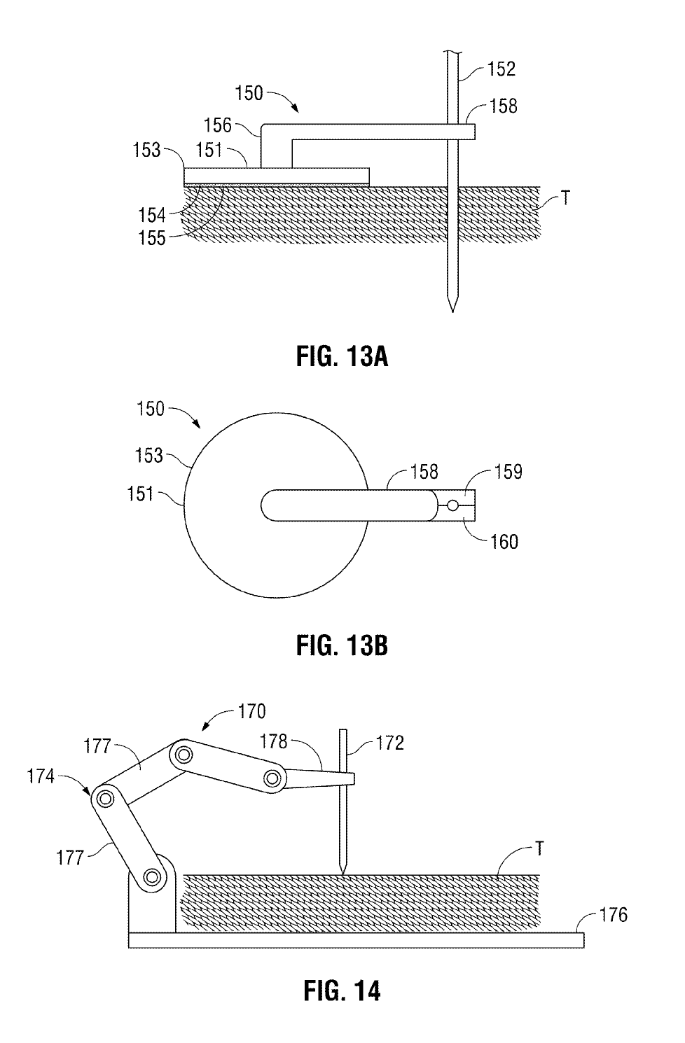

FIGS. 13A and 13B show an ablation probe fixation apparatus 150 for securing an ablation probe 152 within the tissue “T” (FIG. 13A). The apparatus 150 includes a base 151 having a top surface 153 and a skin-contacting bottom surface 154. The base 151 may have any suitable shape such as oval, round, rectangular, polygonal, etc. The base 151 also includes an adhesive layer 155 disposed on the bottom surface 154 thereof as shown in FIG. 13A. The apparatus 150 also includes a fixation post 156 that may be integral with the base 151 or may be formed from a separate structure and then attached thereto.

With reference to FIG. 13B, the apparatus 150 also includes a clamp 158 coupled to the post 156. The clamp 158 may be substantially similar to the clamp 58 of FIGS. 5A and 5B. The clamp 158 extends over the base 151 such that the clamp 158 is disposed over tissue “T.” The post 156 may be rotatably coupled to the base 151 allowing the clamp 158 to be rotated about the post 156. The clamp 158 may include two opposing clamping members 159 and 160 (FIG. 13B) configured to clamp the probe 152. Each of the clamping members 159 and 160 may include a high friction surface (not explicitly shown) formed from a high friction compressible material (e.g., rubber, foam, etc.) to lessen the force applied to the probe 152 and may also include an adhesive layer to provide additional fixation reliability.

During operation, the apparatus 150 is secured against the tissue “T” via the adhesive layer 155. In one embodiment, a protective film may be disposed over the adhesive layer 155 to protect the adhesive prior to use. Thereafter, the opposing clamping members 159 and 160 are opened allowing the probe 152 to be inserted therebetween and into the tissue “T” (FIG. 13A). Once the probe 152 is in a desired location, the opposing clamping members 159 and 160 are closed clamping the probe 152 in place.

In one embodiment, the apparatus 150 may include multiple clamps 158 disposed on the post 156 to provide for insertion and fixation of multiple probes 152. In another embodiment, the apparatus 150 may include one or more skin temperature monitoring devices, such as thermal probes, thermocouples, thermistors, optical fibers and the like, to monitor skin surface temperature.

FIG. 14 shows another embodiment of an ablation probe fixation apparatus 170 for securing an ablation probe 172 within the tissue “T” (e.g., patient) resting on an operating surface 176 (e.g., operating table). The apparatus 170 includes a clamp arm 174 secured to the operating surface 176. The clamp arm 174 includes multiple linkages 177 and a clamp 178 for clamping the ablation probe 172. The multiple linkages 177 may be biased with respect to each other allowing for spatial adjustment of the clamp 178. The clamp 178 may include two opposing clamping members) configured to clamp the probe 172. In one embodiment, the linkages 177 may be robotically controlled. The linkages 177 may also be locked once a desired position of the clamp 178 is achieved.

During operation, the clamp arm 174 is positioned above the tissue “T” at a desired location. The opposing jaw members of the clamp 178 are then opened to allow for the probe 172 to be inserted therebetween and into the tissue “T.” The clamp arm 174 may be adjusted and the linkages 177 are then locked to prevent movement of the clamp 178. Since the clamp arm 174 is secured to the operating surface 176 and not the tissue “T,” any movement of the patient is not translated to the probe 172 thereby maintaining the probe 172 within the tissue “T” throughout the procedure.

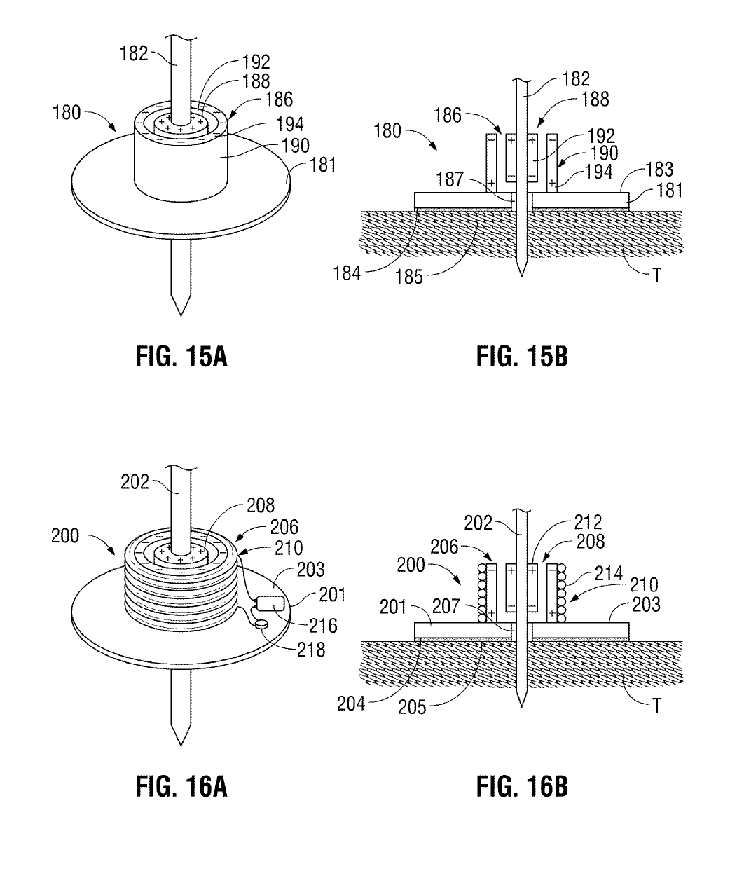

FIGS. 15A-15B show an ablation probe fixation apparatus 180 for securing an ablation probe 182 within the tissue “T” (FIG. 15B). The apparatus 180 includes a base 181 having a top surface 183 and a skin-contacting bottom surface 184. The base 181 may have any suitable shape such as oval, round, rectangular, polygonal, etc. The base 181 also includes an adhesive layer 185 disposed on the bottom surface 184 thereof as shown in FIG. 15B. The base 181 also includes an aperture 187 for insertion of the probe 182 therethrough and into the tissue “T” (FIG. 15B).

As shown in FIG. 15A, the apparatus 180 also includes a fixation assembly 186. The fixation assembly 186 includes a first magnetic coupling 188 disposed on the probe 182 and a second magnetic coupling 190 disposed on the base 181. The first and second magnetic couplings 188 and 190 include statically polarized magnets 192 and 194 respectively. The magnets 192 and 194 are oriented in opposing polarization (e.g., poles of the magnet 192 are disposed opposite their counterpart poles of the magnet 194).

During operation, the apparatus 180 is secured against the tissue “T” via the adhesive layer 185. In one embodiment, a protective film may be disposed over the adhesive layer 185 to protect the adhesive prior to use. Thereafter, the first magnetic coupling 188 is inserted over the probe 182. In another embodiment, the magnetic coupling 188 may include a first half-shell and a second half-shell (not explicitly shown) joined together by a hinge (e.g., a living hinge) that may be clamped around the probe 182. The magnetic coupling 188 is disposed on the probe 182 at a predetermined location such that the probe 182 penetrates the tissue “T” to a desired depth. More specifically, the thickness of the magnetic coupling 188 is larger than the diameter of the aperture 187 (FIG. 15B). This allows the magnetic coupling 188 to act as a stopper, thereby holding the probe 182 at the desired depth. The probe 182 along with the magnetic coupling 188 is inserted into the tissue “T” through base 181, during which the oppositely oriented magnetic couplings 188 and 190 secure the probe 182 within the apparatus 180 due to the opposing acting magnetic fields. In one embodiment, the apparatus 180 may include one or more skin temperature monitoring devices, such as thermal probes, thermocouples, thermistors, optical fibers and the like, to monitor skin surface temperature (FIG. 3A).

FIGS. 16A-16B show an ablation probe fixation apparatus 200 for securing an ablation probe 202 within the tissue “T” (FIG. 16B). The apparatus 200 includes a base 201 having a top surface 203 and a skin-contacting bottom surface 204. The base 201 may have any suitable shape such as oval, round, rectangular, polygonal, etc. The base 201 also includes an adhesive layer 205 disposed on the bottom surface 204 thereof as shown in FIG. 16B. The base 201 also includes an aperture 207 for insertion of the probe 182 therethrough and into the tissue “T” (FIG. 16B).

As shown in FIG. 16A, the apparatus 200 also includes a fixation assembly 206. The fixation assembly 206 includes a first magnetic coupling 208 disposed on the probe 202 and a second magnetic coupling 210 disposed on the base 201. With reference to FIG. 16B, the first magnetic coupling 208 includes a statically polarized magnet 212 and the second magnetic coupling 210 includes an electromagnet 214 (e.g., a solenoid). As shown in FIG. 16A, the electromagnet 214 is coupled to a power source 216 and a switch 218. When the electromagnet 218 is powered (e.g., the switch 218 is toggled and the power source 216 supplies the current through the electromagnet), the electromagnet 218 is polarized. The magnet 212 and the electromagnet 214 are oriented in opposing polarization (e.g., poles of the magnet 212 are disposed opposite their counterpart poles of the electromagnet 214).

During operation, the apparatus 200 is secured against the tissue “T” via the adhesive layer 205. In one embodiment, a protective film may be disposed over the adhesive layer 205 to protect the adhesive prior to use. Thereafter, the first magnetic coupling 208 is inserted over the probe 202. In another embodiment, the magnetic coupling 208 may include a first half-shell and a second half-shell (not explicitly shown) joined together by a hinge (e.g., a living hinge) that may be clamped around the probe 202. The magnetic coupling 208 is disposed on the probe 202 at a predetermined location such that the probe 202 penetrates the tissue “T” to a desired depth. More specifically, the thickness of the magnetic coupling 208 is larger than the diameter of the aperture 207. This allows the magnetic coupling 208 to act as a stopper, thereby holding the probe 202 at the desired depth. The probe 202 along with the magnetic coupling 208 is inserted into the tissue “T” through base 201.

The switch 218 is toggled and the electromagnet 214 is energized thereby creating a magnetic field. Due to opposite polarization of the magnet 212 and the electromagnet 214, the probe 202 is secured within the apparatus 200. Once the ablation procedure is complete, the switch 218 may be toggled to terminate the supply of current to the electromagnet 214, thereby terminating the magnetic field and allowing for withdrawal of the probe 202 from the tissue “T.” In one embodiment, the apparatus 200 may include one or more skin temperature monitoring devices, such as thermal probes, thermocouples, thermistors, optical fibers and the like, to monitor skin surface temperature.

The described embodiments of the present disclosure are intended to be illustrative rather than restrictive, and are not intended to represent every embodiment of the present disclosure. Various modifications and variations can be made without departing from the spirit or scope of the disclosure as set forth in the following claims both literally and in equivalents recognized in law.

1-15. (canceled)

16. An ablation probe fixation apparatus for securing an ablation probe to tissue, the ablation probe fixation apparatus comprising:
a base having a top surface and a bottom surface opposite the top surface, the base defining an aperture through the top surface and the bottom surface;
a support shaft projecting from the top surface of the base and having an inner surface defining an aperture configured for receipt of an ablation probe; and
a camming member rotatably coupled to the support shaft and having a camming surface, the camming member rotatable relative to the support shaft between an open configuration, wherein an ablation probe is receivable within the aperture of the support shaft, and a closed configuration, wherein the camming surface of the camming member is configured to releasably engage an ablation probe received within the aperture of the support shaft.

17. The ablation probe fixation apparatus according to claim 16, wherein the camming member is resiliently biased toward the closed configuration.

18. The ablation probe fixation apparatus according to claim 16, wherein the inner surface of the support shaft has a portion that faces the camming surface of the camming member, the camming surface of the camming member being disposed closer to the portion of the inner surface of the support shaft when the camming member is in the closed configuration than when the camming member is in the open configuration.

19. The ablation probe fixation apparatus according to claim 16, wherein the support shaft has a circular configuration.

20. The ablation probe fixation apparatus according to claim 16, wherein the support shaft extends perpendicularly from the top surface of the base.

21. The ablation probe fixation apparatus according to claim 16, wherein at least one of the inner surface of the support shaft or the camming surface of the camming member is formed from a high friction compressible material.

22. The ablation probe fixation apparatus according to claim 16, further comprising a biasing member disposed between the support shaft and the camming member, the biasing member configured to resiliently bias the camming member toward the closed configuration.

23. The ablation probe fixation apparatus according to claim 16, wherein the base has an adhesive layer disposed on the bottom surface.

24. The ablation probe fixation apparatus according to claim 16, wherein the support shaft includes:
a first end rotatably connected to a first lateral surface of the camming member; and
a second end rotatably connected to a second lateral surface of the camming member.

25. An ablation probe fixation apparatus for securing an ablation probe to tissue, the ablation probe fixation apparatus comprising:
a base having a top surface and a bottom surface opposite the top surface, the base defining an aperture through the top surface and the bottom surface;
a first camming member disposed adjacent a first side of the aperture and rotatably connected to the base, the first camming member having a camming surface; and
a second camming member disposed adjacent a second side of the aperture and rotatably connected to the base, the second camming member having a camming surface facing the camming surface of the first camming member, the first and second camming members rotatable between an open configuration, wherein an ablation probe is receivable within the aperture of the base, and a closed configuration, wherein the camming surface of each of the first and second camming members is configured to releasably engage an ablation probe received within the aperture of the base.

26. The ablation probe fixation apparatus according to claim 25, wherein the first and second camming members are resiliently biased toward the second configuration.

27. The ablation probe fixation apparatus according to claim 25, wherein the camming surface of the first camming member is closer to the camming surface of the second camming member when the first and second camming members are in the closed configuration than when in the open configuration.

28. The ablation probe fixation apparatus according to claim 25, wherein at least one of the camming surface of the first camming member or the camming surface of the second camming member is formed from a high friction compressible material.

29. The ablation probe fixation apparatus according to claim 25, wherein the base has an adhesive layer disposed on the bottom surface.
